(19) United States
(12) Patent Application Publication
(10) Pub. No.: US 2007/0028922 A1
(43) Pub. Date: Feb. 8, 2007

(54) Breathing bag including integrally formed connector and distensible portions

(75) Inventors: Richard Kennedy, Mine Hill, NJ (US); Jack H. Britten, Blairstown, NJ (US); Mark E. Woelfel, Oak Ridge, NJ (US)

Correspondence Address: R. GALE RHODES, ESQ. / MOSER IP LAW GROUP, 1040 BROAD STREET, 2ND FLOOR, SHREWSBURY, NJ, 07702, US

(73) Assignee: Vital Signs, Inc.

(21) Appl. No.: 11/545,261

(22) Filed: Oct. 10, 2006

Related U.S. Application Data
(63) Continuation-in-part of application No. 11/355,366, filed on Feb. 16, 2006.
Application No. 11/355,366 is a continuation of application No. 11/037,438, filed on Jan. 18, 2005, now abandoned.
Application No. 11/037,438 is a continuation of application No. 10/843,769, filed on May 12, 2004, now abandoned.
Application No. 10/843,769 is a division of application No. 10/176,906, filed on Jun. 21, 2002, now Pat. No. 6,878,335.

(51) Int. Cl. A61M 16/00

(52) U.S. Cl. 128/205.14

(57) ABSTRACT

A breathing bag including integrally formed connector portion, distensible bag portion and an inverted truncated conical portion intermediate the connector portion and the distensible bag portion, the connector portion being sufficiently thick or rigid to be connected directly to a male fitting.

### CROSS-REFERENCE TO RELATED APPLICATIONS

This application is a continuation-in-part application of pending U.S. patent application Ser. No. 11/355,366, filed Feb. 16, 2006, entitled BREATHING BAG INCLUDING INTEGRALLY FORMED CONNECTOR AND DISTENSIBLE PORTIONS, which is a continuation of U.S. patent application Ser. No. 11/037,438, filed Jan. 18, 2005 (now abandoned), entitled BREATHING BAG INCLUDING INTEGRALLY FORMED CONNECTOR AND DISTENSIBLE PORTIONS, which is a continuation application of U.S. patent application Ser. No. 10/843,769, filed May 12, 2004 (now abandoned), entitled BREATHING BAG AND BREATHING BAG MANUFACTURED BY A PROCESS, which is a divisional application of U.S. patent application Ser. No. 10/176,906, filed Jun. 21, 2002 (now U.S. Pat. No. 6,878,335), entitled PROCESS OF MANUFACTURING A BREATHING BAG AND BREATHING BAG MANUFACTURED BY SUCH PROCESS, Jack H. Britten, et al., inventors, and assigned to the same assignee as this continuation application.

### BACKGROUND OF THE INVENTION

This invention relates to a breathing bag including connector and distensible portions.

Breathing bags are well known to the medical arts for delivering, or assisting in delivering, gas to a patient such as anesthesia gas or a breathing gas such as oxygen or oxygen-enriched air. Typical prior art breathing bags are illustrated diagrammatically in FIGS. 1A and 1B, FIGS. 2A and 2B, FIGS. 3A, 3B, 3C and 3D and FIGS. 4A, 4B and 4C.

These prior art breathing bags typically include connector and distensible portions which are fluted, and hence, such prior art breathing bags are typically referred to as fluted breathing bags or breathing bags having a fluted distensible portion.

In use, for example, in an anesthesia circuit for supplying anesthesia gas to a patient's lungs, the prior art breathing bag of FIGS. 3A, B and C includes a connector portion 3d and a fluted distensible portion 3e which is filled with pressurized anesthesia gas, pressurized to the normal operating pressure, and the fluted distensible portion 3e, shown in its manufactured shape in cross-section in FIG. 3C, is distended, expanded or stretched, outwardly into the circular cross-section shape S1 shown in FIG. 3D which is the pressurized anesthesia gas filled normal operating state of the distensible breathing bag portion. Thereafter, the anesthesiologist alternately squeezes and relaxes the distensible portion 3e to supply the appropriate amount of anesthesia gas to the lungs of the patient. The thinness of the distensible portion 3e permits it to initially distend or expand outwardly from the manufactured state or shape shown in FIG. 3C into the normal operating state S1 shown in FIG. 3D and to re-distend, or re-expand, into the normal operating state S1 after the anesthesiologist has relaxed the squeezing in supplying anesthesia gas to the patient. In the event an unintended increase in anesthesia gas pressure occurs in t he anesthesia circuit, such as an unintended anesthesia gas pressure surge, the distensible portion 3e is also sufficiently thin to permit the distensible portion to further distend, expand or stretch, and outwardly into the further distended state S2 shown in FIG. 3D. This further distension of the distensible portion 3e into state or shape S2 permits it to increase its volume and receive or accommodate the unintended anesthesia gas pressure increase and prevent such pressure increase from reaching and possibly damaging the patient's lungs. Accordingly, it will be understood that the term distensible portion as used hereinafter, and in the appended claims, is defined to mean a breathing bag distensible portion that is sufficiently thin to distend or expand outwardly from its manufactured state into a pressurized gas filled normal operating state and to contract and re-distend into its normal operating state upon being alternately squeezed and relaxed, and to further distend or expand outwardly into a further distended state to increase its volume and receive and accommodate an increase in gas pressure.

Typically, in the prior art, such breathing bags are made by the dipping process much like the process used for producing a candle. A form is made in the shape of the breathing bag desired and the form is dipped repeatedly into a fluid suitable for producing a breathing bag such as, for example, a latex or non-latex fluid. Such process is typically slow, unwantedly expensive and lends itself to imperfections.

Further, the typical prior art breathing bag made by the aforenoted dipping process and has a relatively thin wall thickness such that the connector portion is insufficiently stiff or rigid to connect, for example, to a standard 22 mm tapered male fitting or a non-tapered tubular or hollow cylindrical fitting. More particularly and referring to FIGS. 5 and 5A, these FIGS. show a typical prior art breathing bag made by the above-noted dipping process and each breathing bag includes a distensible portion 2 and a generally cylindrical neck or connector portion 4. Having been made by the above-noted dipping process, the distensible portion 2 and the neck or connector portion 4 are typically 0.010-0.015 inch thick. Such thickness means that the neck portions 4 are insufficiently stiff or rigid to be connected directly to one of the above-noted male fittings. Hence, as is further known to the art, these prior art breathing bags made by the dipping process require the addition of a separate cylindrical bushing such as the bushings 5 and 6 shown respectively in FIGS. 5 and 5A to be inserted into the neck or connector portion to provide the required stiffness or rigidity to permit these breathing bags, namely the connector portion, to be connected to the above-noted male fittings. Typically, such bushings 5 and 6 are made from polyvinyl chloride and have a wall thickness of about 0.125 inch. Although such bushings 5 and 6 are sufficiently rigid or stiff to permit the connector portions of the breathing bags to be connected to one of the above-noted fittings, such bushings are still sufficiently soft to permit the bushings to slidably or wedgedly engage the above-noted male fittings. As will be noted from FIG. 5, the bushing 5 has an inwardly tapered internal wall for being fitted to a tapered male fitting in a sliding or wedged air-tight engagement, and the bushing 6 in FIG. 5A has a straight or cylindrical internal wall for air-tight sliding or wedged engagement with a tubular or cylindrical fitting. Further, as will be understood from FIG. 5A, to retain the bushings 5 and 6 in the neck or connector portion 4 of these breathing bags, an additional element is included such as the surrounding retaining ring or band 7 shown in FIG. 5A which surrounds the bag neck portion 4 sufficiently tightly so as to retain the bushing 6 in the neck portion of the breathing bag. At least certain of the bushings are provided with anti-occlusion members for preventing occlusion of the internal airway between the distensible and connector portions in the event the distensible portion is bent acutely with respect to the connector portion. As is further known to the art, the requirement of these additional bushings, the retaining bands, and the manufacturing steps required to insert and retain the bushings in the breathing bags further add unwanted cost and expense to the manufacture of the typical prior art breathing bag.

Accordingly, there is a need in the breathing bag art for a new and improved breathing bag which has a neck or connector portion formed integrally with the distensible portion and which neck or connector portion is sufficiently thick, stiff or rigid, to permit it to connect directly to a male fitting of the types noted above, the distensible portion may be fluted.

### SUMMARY OF THE INVENTION

A breathing bag including integrally formed connector portion, distensible bag portion and an inverted truncated conical portion intermediate the connector portion and the distensible bag portion, the connector portion being sufficiently thick or rigid to permit the connector portion to be connected directly to a male fitting.

### DESCRIPTION OF THE DRAWINGS

FIGS. 1A and B illustrate, respectively, a side elevational view of a double fluted standard prior art breathing bag with a nipple, FIG. 1B shows the flutes and is rotated approximately 45′ with respect to FIG. 1A;

FIGS. 2A and 2B show a bag similar to that shown in FIGS. 1A and 1B except the prior art bag in FIGS. 2A and 2B does not include the nipple;

FIG. 3A is a side elevational view of a double fluted prior art paddle bag with a nipple and FIG. 3B shows the bag of FIG. 3A rotated approximately 45° and illustrates the double flute, FIG. 3C is a cross-sectional view taken generally along the line 3-3 in FIG. 3B in the direction of the arrows and illustrates more clearly the double fluting;

FIG. 3D is a double cross-sectional view taken generally along the line 3-3 in FIG. 3B but showing the distensible portion of the breathing bag distended into the normal operating state upon receiving pressurized gas and further distended into a further distended state to accommodate an increase in gas pressure.

FIG. 4A is a side elevational view of a standard prior art breathing bag with triple flutes which flutes are shown in FIG. 4B which FIG. is rotated approximately 45° with respect to FIG. 4A, this bag also is provided with a nipple, FIG. 4C is a diagrammatical cross-sectional view taken generally along the line 4-4 in FIG. 4B in the direction of the arrows and shows more clearly the triple fluting;

FIGS. 5 and 5A, illustrate, in perspective, still further breathing bags known to the prior art;

FIG. 6 is a side elevational view of a breathing bag embodying the present invention and made by the process of the present invention;

FIG. 6A is an enlarged view in cross-section of the encircled portion of FIG. 6;

FIGS. 7-9 illustrate, diagrammatically, the process of the present invention for manufacturing a breathing bag and also illustrate, diagrammatically, apparatus suitable for practicing such process;

FIG. 8A is a diagrammatical illustration of a portion of a combination blowing and forming member which may be included in the apparatus for practicing the process of the present invention;

FIG. 9A is a cross-section taken generally along the line 9A-9A in FIG. 9 and in the direction of the arrows;

FIGS. 10-12 illustrate, diagrammatically, a second embodiment of the process of the present invention for manufacturing a breathing bag and further illustrate, diagrammatically, apparatus suitable for practicing such process;

FIG. 13 is a partial view, partially in cross-section, of the connector portion of a breathing bag adhered to a combination blowing and forming member;

FIG. 14 is a partial external view of an additional embodiment of the breathing bag of the present invention;

FIG. 15 is a vertical cross-sectional view of the breathing bag of FIG. 14 taken generally along the line 15-15 in FIG. 14 and in the direction of the arrows;

FIG. 16 is a view, partially in cross-section, taken generally along the line 16-16 in FIG. 14 and in the direction of the arrows;

FIG. 17 is a view similar to FIG. 9 and illustrates apparatus suitable for practicing the process for manufacturing the breathing bag of FIG. 14;

FIG. 18 is a partial view, partially in cross-section, of the connector portion of the breathing bag of FIG. 14 adhered to the combination blowing and forming member of the apparatus shown in FIG. 17;

FIG. 19 is a perspective view of a pair of engaging and transfer members suitable for holding the connector portion of the breathing bag shown in FIG. 18 as the combination blowing and forming member is withdrawn from the connector portion to separate the breathing bag from the combination blowing and forming member and which members can be used to transfer the separated breathing bag to a further station;

FIG. 20 is a view, partially in cross-section, showing the members of FIG. 19 in engagement with the connector portion of the breathing bag;

FIG. 21 is a view, partially on cross-section, showing the members of FIG. 19 holding the connector portion of the breathing bag stationary as the combinations blowing and forming member is withdrawn; and

FIG. 22 is a diagrammatical view illustrating a breathing bag bent at an angle with respect to a male fitting.

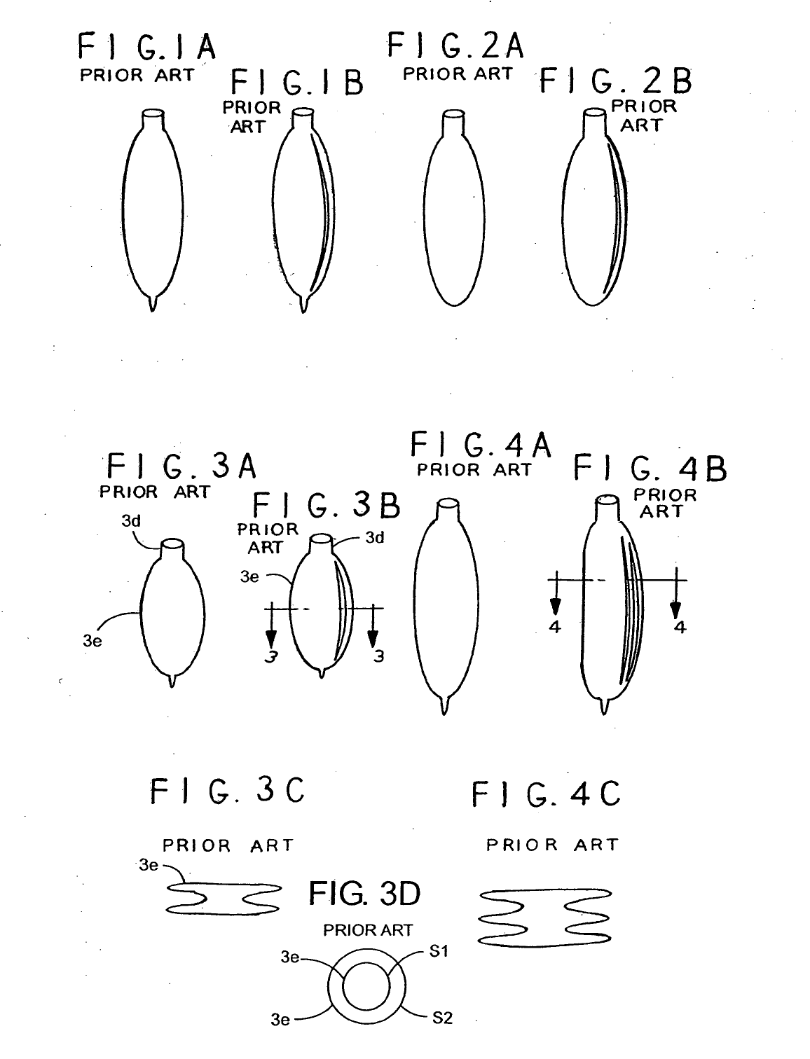

It will be generally understood that the opposite sides of the bags shown in FIGS. 1B, 2B, 3B and 4B are also provided with flutes in the same manner as the sides of these bags shown in these FIGS.

### DETAILED DESCRIPTION

A breathing bag embodying the present invention is illustrated diagrammatically in FIGS. 6 and 6A and indicated by alphabetical designation B. Breathing bag B includes a generally cylindrical connector portion C and a distensible portion D. The connector portion C is typically sized to connect directly to an ISO 22 mm male fitting and the distensible bag D expands and contracts in the process of delivering, or assisting in delivering, gas to the patient as noted above. As will be better understood from FIG. 6A, the connector portion C has a greater thickness than the thickness of the distensible portion D; the greater thickness of the connector portion C provides it with the strength or rigidity to serve its above-noted connector function. For illustration the thickness of the connector portion C has been exaggerated with respect to the thickness of the distensible portion D in FIG. 6A. In one embodiment, the distensible portion D had a thickness or wall thickness T1 of about 0.015-0.020 inch and the neck portion C had a thickness or wall thickness T2 of about 0.125 inch. In accordance with the teaching of the present invention, the greater thickness T2 of the neck or connector portion C provides sufficient rigidity or stiffness to permit such neck portion C to connect the breathing bag B, FIGS. 6 and 6A, directly to the above-noted male fittings, and yet be sufficiently flexible or soft that it can engage such fittings in a sliding or wedged air-tight engagement.

As shown in FIG. 6A, an internal airway is provided between the distensible and connector bag portions. In the breathing bag of the present invention internal anti-occlusion ridges E (FIG. 6A) may be molded into the integral connection between the distensible and connector portions to maintain a positive airway between these portions. The distensible portion D, FIG. 6, of the breathing bag B may be a fluted distensible portion as indicted by the area F.

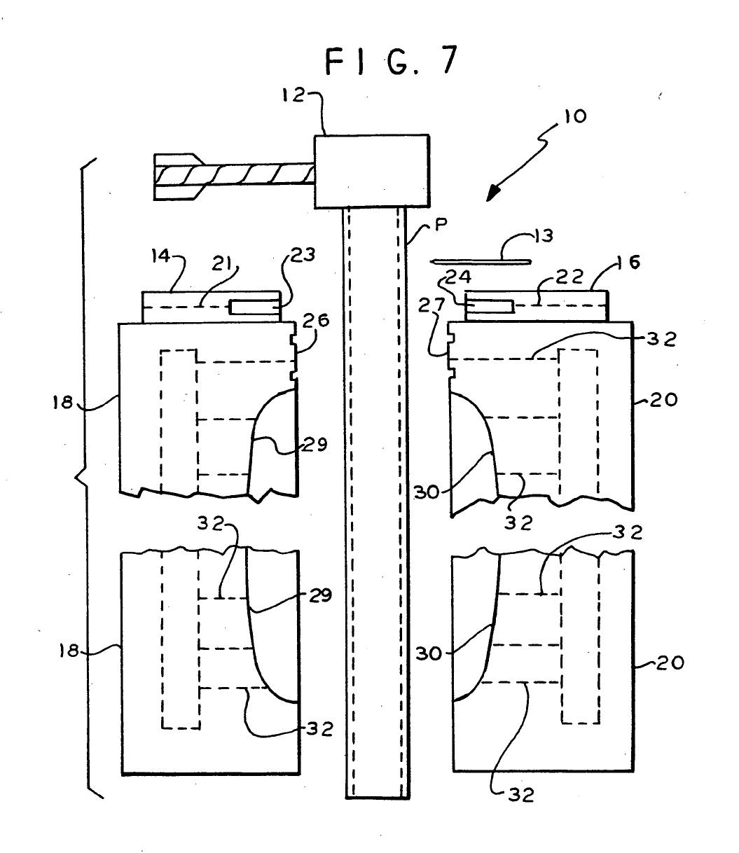

The process for manufacturing a breathing bag according to the present invention is illustrated diagrammatically in FIGS. 7-9. Such process will be described by way of example with respect to a process for manufacturing the breathing bag B illustrated in FIGS. 6, and 6A. Also illustrated diagrammatically in FIGS. 7-9 are apparatus indicated by general numerical designation 10 suitable for practicing such process. Apparatus 10 may include an extruder 12 for extruding a hollow tubular parison of thermoplastic material suitable for making the breathing bag B. As known to the art, a parison is a hollow tubular length of relatively molten, or semi-molten, thermoplastic material. The apparatus 10 further includes a hot cutting knife 13 for cutting the parison, a pair of holding jaws 14 and 16, and a pair of opposed main molds 18 and 20. The holding jaws and the main molds are mounted for sliding reciprocal movement toward and away from each other and function in the manner known to the art.

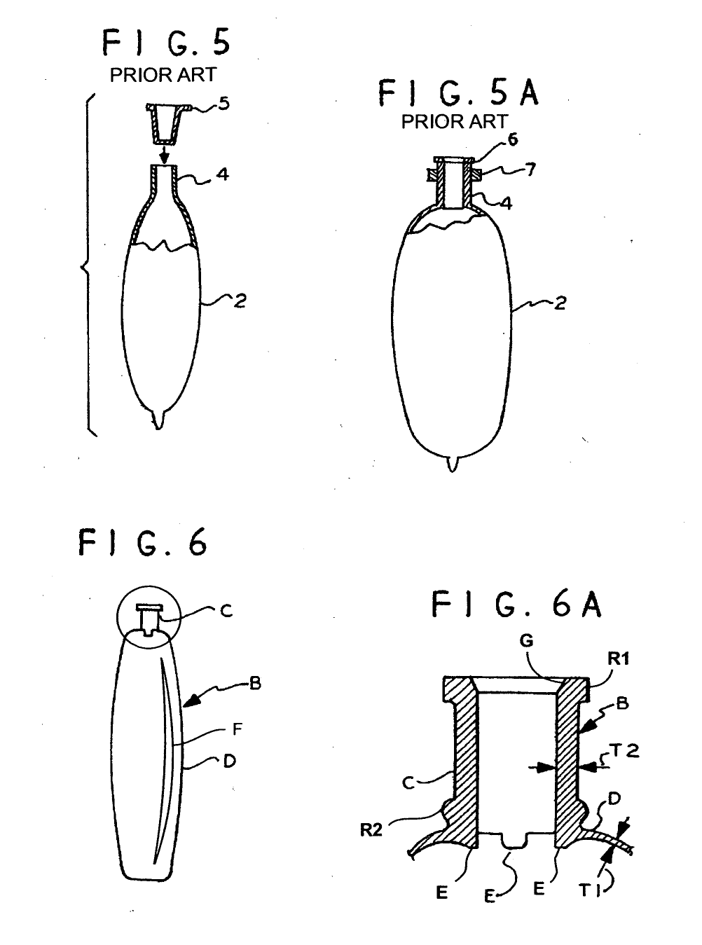

The main molds 18 and 20 are provided with connector forming portions 26 and 27 for forming the exterior of the connector portion C of the breathing bag B (FIGS. 5, 6 and 6A) and are further provided with opposed centrally formed mold cavities 29 and 30 for forming the distensible portion D of the breathing bag B. The forming portions 26 and 27 of the main molds 18 and 20 are complementary in shape to the exterior of the connector portion C of the breathing bag B of FIGS. 5, 6 and 6A and the mold cavities 29 and 30 are complementary in shape to the distensible portion D of the breathing bag B.

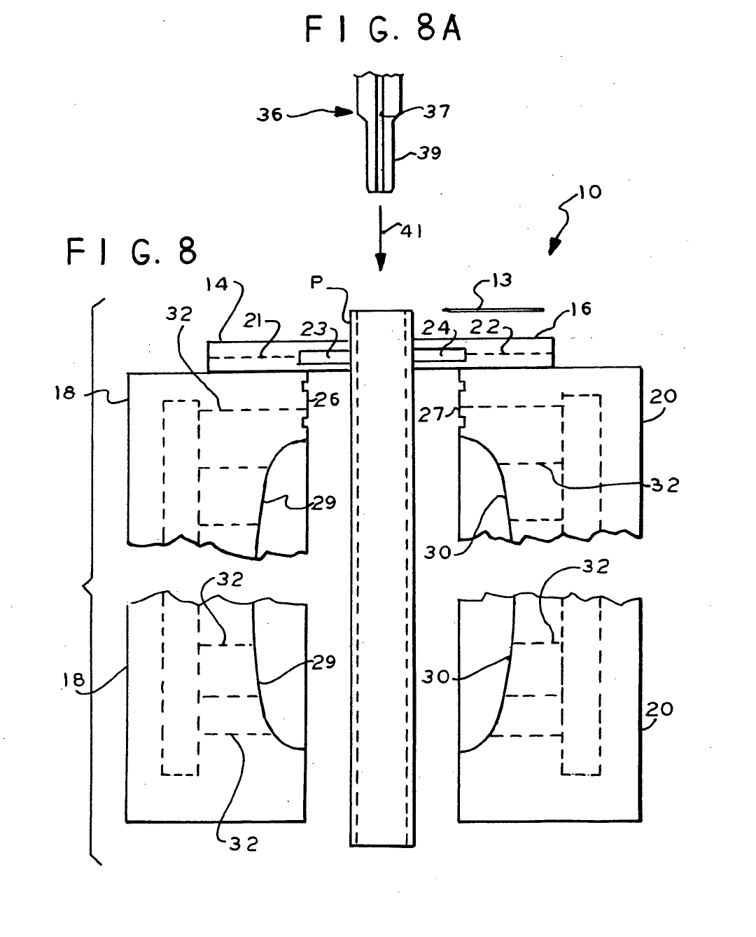

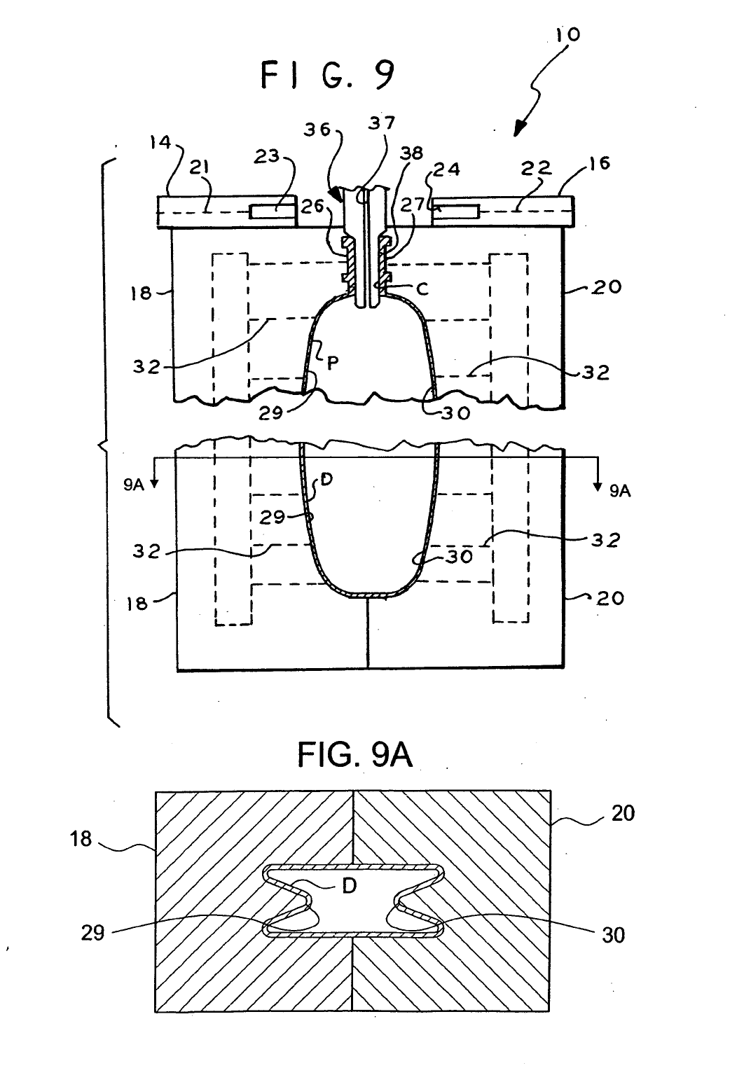

Referring to FIG. 8A, the apparatus 10 may further include a combination blowing and forming member indicated by general numerical designation 36. The member 36 is provided with a centrally formed passageway 37 through which pressurized air is blown into the interior of the parison to expand and force a portion of the parison wall into the mold cavities 29 and 30 (FIG. 9) and is further provided with an external surface 38 for forming the internal surface, or hollow interior portion of the connector portion C of the breathing bag B (FIG. 6A). It will be understood that such external surface 38 is shaped complementarily to the external shape of the male fitting to which the connector portion C, FIG. 6A, of the breathing bag B will engage in a sliding or wedged air-tight fit. Accordingly, such external surface 38 of the combination blowing and forming member 36 may be provided with a shape so as to provide the connector portion C, FIG. 6A, or the hollow interior portion thereof, with a tapered shape or a non-tapered straight or cylindrical shape for receiving either a tapered male fitting or a non-tapered male fitting in a sliding or wedged air-tight engagement.

The process for manufacturing a breathing bag in accordance with the present invention, and with regard to the breathing bag B of FIGS. 5 and 6 by way of example, will now be described in reference to FIGS. 7-9. The parison P, FIG. 7, is extruded downwardly by the extruder 12 between the pairs of open holding jaws 14 and 16 and main molds 18 and 20 as shown in FIG. 7. As shown in FIG. 8, the holding jaws 14 and 16 are advanced into engagement with the upper portion of the parison P, vacuum is drawn in the vacuum lines 21 and 22, and the cavities 23 and 24 to secure the parison P between the open main molds 18 and 20. The hot cutting knife 13 cuts off the upper portion of the parison P and the extruder 12 (FIG. 7) is suitably indexed away. The combination blowing and forming member 36 is then suitably indexed into position over the open upper portion of the parison P, FIG. 8, and is inserted into the upper portion of the parison P as indicated by the arrow 41 in FIG. 8A.

As further illustrated in FIG. 9, the main molds 18 and 20 are advanced toward an engagement with each other to cause the connector forming portions 26 and 27 and the outer surface 38 of the combination blowing and forming member 36 to compression form or mold therebetween the upper portion of the parison P into the generally cylindrical connector portion C of the breathing bag B (FIG. 6). Pressurized air is blown into the interior of the parison P through the passageway 37 formed in the combination blowing and forming member 36 to expand and force the lower portion of the parison P into engagement with the surfaces defining the mold cavities 29 and 30 to thereby blow mold the distensible portion D of the breathing bag B (FIG. 6).

Referring to FIG. 9A, it will be understood that the mold surfaces 29 and 30 of the molds 18 and 20 may be suitably shaped to provide the distensible portion D of the breathing bag B of the present invention with flutes, such as the double flutes of the prior art breathing bag illustrated diagrammatically in FIG. 3C and described above. Hence, it will be understood that the breathing bag B of the present invention upon having a fluted distensible portion D may be referred to as a fluted breathing bag or a breathing bag having a fluted distensible portion.

The combination blowing and forming member 36 is withdrawn and the main molds 18 and 20 are then retracted or opened and the now formed breathing bag B, of FIG. 6, has been manufactured in accordance with the manufacturing process of the present invention.

It will be understood in accordance with the further teachings of the present invention that if desired or required, the blow-molding step may be accompanied by a vacuum assisted molding step to further enhance the molding of the distensible portion D of the breathing bag B of FIG. 6. In such event, this additional method step will be practiced by providing the main molds 18 and 20, FIGS. 8-9, with vacuum lines or channels 32 for applying vacuum to the lower portion of the parison P (FIG. 9) to assist in expanding and forcing such portion of the parison P into the mold cavities 29 and 30.

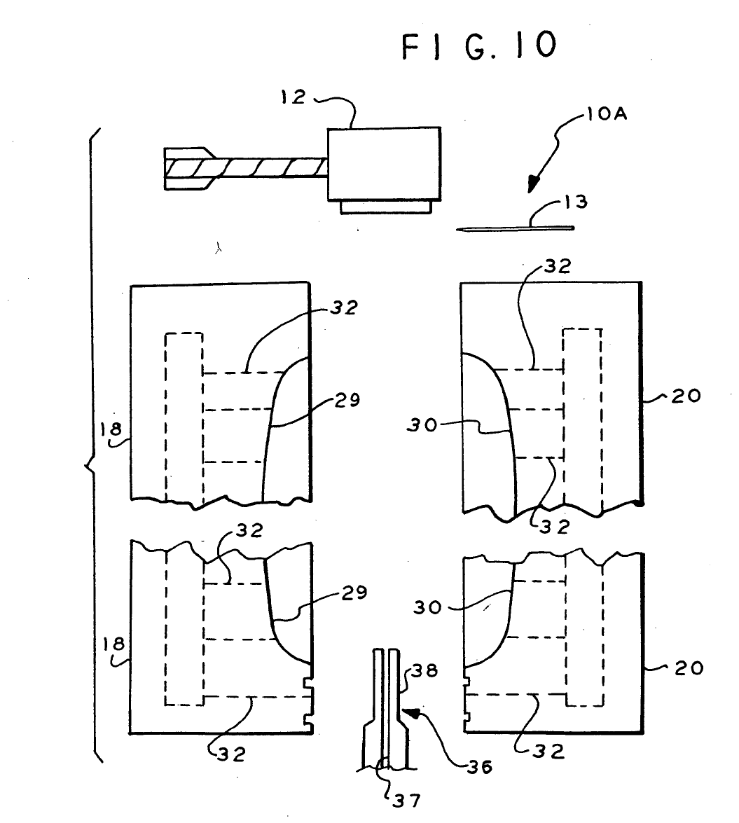

Referring again to FIG. 6A, and as noted above, the generally cylindrical connector portion C of the breathing bag B is thicker than the thickness of the distensible portion D. It has been found that it is further desirable to provide additional parison material in the breathing bag manufacturing process to enhance the forming of the connector portion C of the breathing bag B so as to have a greater thickness than the distensible portion D of the breathing bag. Accordingly, and referring now to FIGS. 10-12, an alternate process for manufacturing a breathing bag according with the present invention is illustrated diagrammatically and apparatus suitable for practicing this alternate process is also illustrated diagrammatically in these figures and indicated by general numerical designation 10A. It will be understood that the components comprising the apparatus 10A in FIGS. 10-12 which are the same as the components comprising the apparatus 10 in FIGS. 7-9 are given the same numerical designations in FIGS. 10-12 and will be understood to perform the same functions.

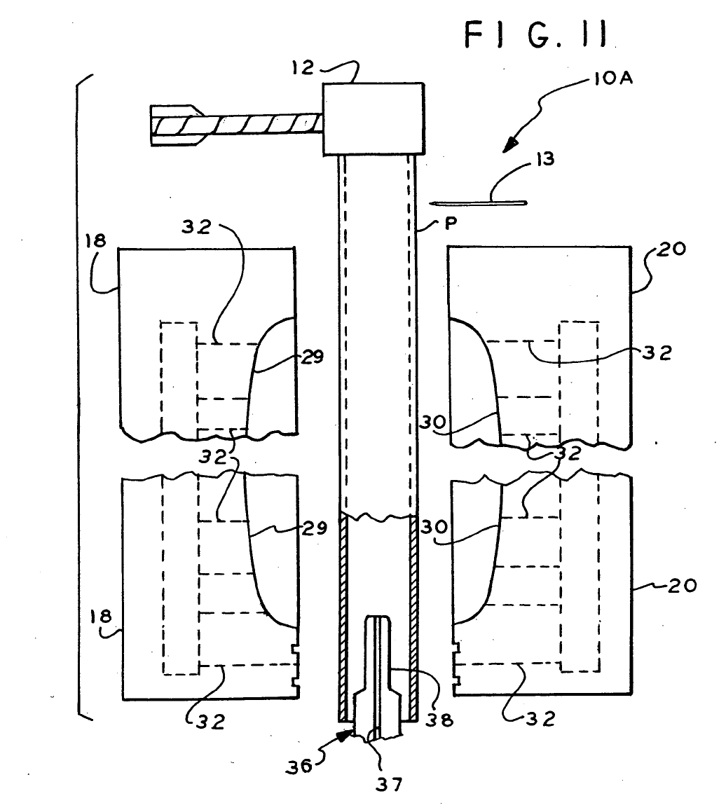

It has been discovered that upon the parison P, FIG. 11, being extruded vertically downwardly, that at least the lower portion of the parison P increases in thickness with respect to the upper portion of the parison under the influence of gravity. It has been further discovered that this thickening of the lower portion of the parison P can be utilized advantageously in forming the generally cylindrical connector portion C of the breathing bag B of FIGS. 6 and 6A. The thickening of the lower portion of the parison P under the influence of gravity is illustrated diagrammatically in FIG. 11 by the cross-sectioned lower portion of such parison.

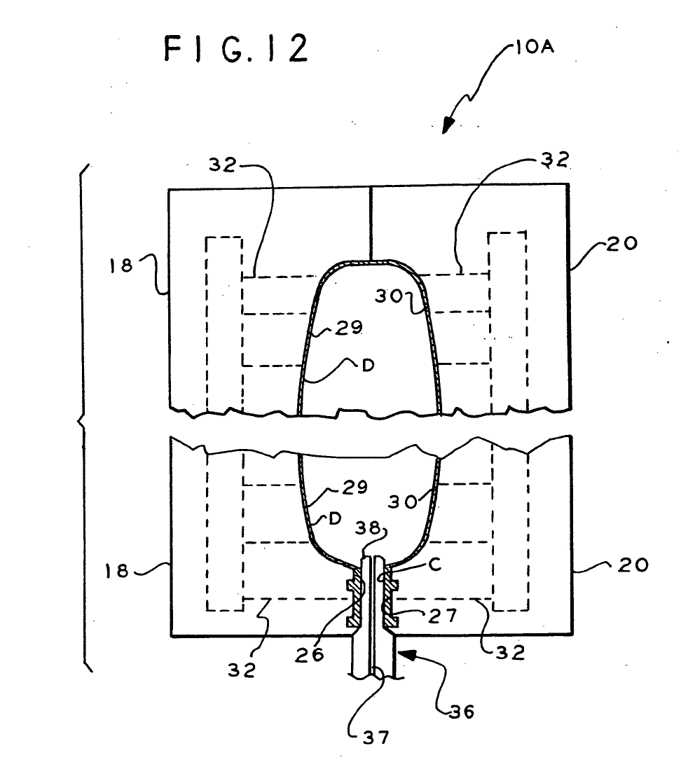

It will be generally understood, that the primary difference between the alternate embodiment process of FIGS. 10-12 and the foregoing described process of FIGS. 7-9 is that the breathing bag in FIGS. 10-12 is formed upside down with respect to the orientation of the breathing bag formed by the process illustrated diagrammatically in FIGS. 7-9. Accordingly, and referring to FIG. 11, the parison P of hollow cylindrical suitable thermoplastic material is extruded vertically downwardly between the open main molds 18 and 20 and the lower portion of such parison is extruded downwardly by the extruder 12 over the combination blowing and forming member 36 and the lower portion of the parison P is allowed to increase in thickness under the influence of gravity. The main molds 18 and 20, FIG. 12, are advanced into engagement with each other, the cutting knife 13 cuts the parison P, the extruder 12 is suitably indexed out of the way, and pressurized air is blown into the interior of the upper portion of the parison P through the combination blowing and forming member 36 and, if desired or required, vacuum is drawn against the exterior of the upper portion of the parison P through the vacuum lines 32 to force the upper portion of the parison P into the mold cavities 29 and 30 and to expand such upper portion of the parison P to form the distensible portion D of the breathing bag B of FIG. 6 by pressure molding or by pressure and vacuum molding. The connector forming portions 26 and 27 of the main molds 18 and 20 and the outer forming surface 38 of the combination blowing and forming member 36 engage the lower portion of increased thickness of the parison P and compression mold or form therebetween the connector portion C of the breathing bag B as illustrated diagrammatically in FIG. 12.

Thereafter, the main molds are retracted into the open position shown in FIGS. 10 and 11 and the combination blowing and forming member 36 is withdrawn and suitably indexed away and the breathing bag B of FIGS. 5 and 6 is removed.

It has been found, by way of example and not by way of limitation, that a thermoplastic material from QST Inc., Ateknor Apex Co., 300 Industrial Park Road, St. Albans, Vt., sold under the trademark MONOPRENE, is suitable for use in the processes of the present invention for manufacturing a breathing bag.

Referring again to the parison P, FIGS. 7, 8 and 11, it will be understood that the tubular parison P may be circular in cross-section, rectangular in cross-section, or of other cross-sectional shapes to enhance the manufacture of the breathing bag in accordance with the processes of the present invention.

Referring again to the breathing bag B of the present invention shown in FIG. 6A, and to the two processes described-above for manufacturing the breathing bag B and described in connection with FIGS. 7-9 and 10-12, the distensible portion D and the integrally formed connector portion C are one piece as shown in FIGS. 6A, 9 and 12.

As shown in FIG. 6A, the outer portion of the connector portion C may be provided with an integrally formed, radially outwardly extending and generally annular ridge R1. The ridge R1 facilitates further increasing the rigidity or stiffness of the connector portion C to further enhance the ability of the connector portion C to connect directly to a male fitting. The ridge R1 also provides a flange for engagement by the hand of a person to facilitate the direct connection of the connector portion C to a male fitting. The ridge R1 may have any transverse cross-sectional shape. In one embodiment, the ridge R1 has a generally rectangular transverse cross-section.

Referring further to FIG. 6A, the lower portion of the connector portion C, adjacent the integral connection between the connector portion C and the distensible portion D, may be provided with an integrally formed, radially outwardly extending and generally annular ridge R2. The ridge R2 provides a feature for gripping by the hand of a person to facilitate the removal of the connector portion C from a male fitting. The ridge R2 may have any transverse cross-sectional shape. In one embodiment, the ridge R2 has a generally semi-circular transverse cross-section.

In addition, as shown in FIG. 6A, the end of the outer portion of the connector portion C may be provided with an internal, outwardly flared, generally annular, beveled surface G. The surface G facilitates the insertion of a male fitting into the connector portion C.

As will be further noted from FIG. 6A, the generally cylindrical connector portion C has a greater thickness T2 at all points therealong than the thickness T1 of the distensible bag portion D. Viewed alternatively, the thickness T1 of the distensible bag portion D is less than the thickness T2 at all points along the connector portion C.

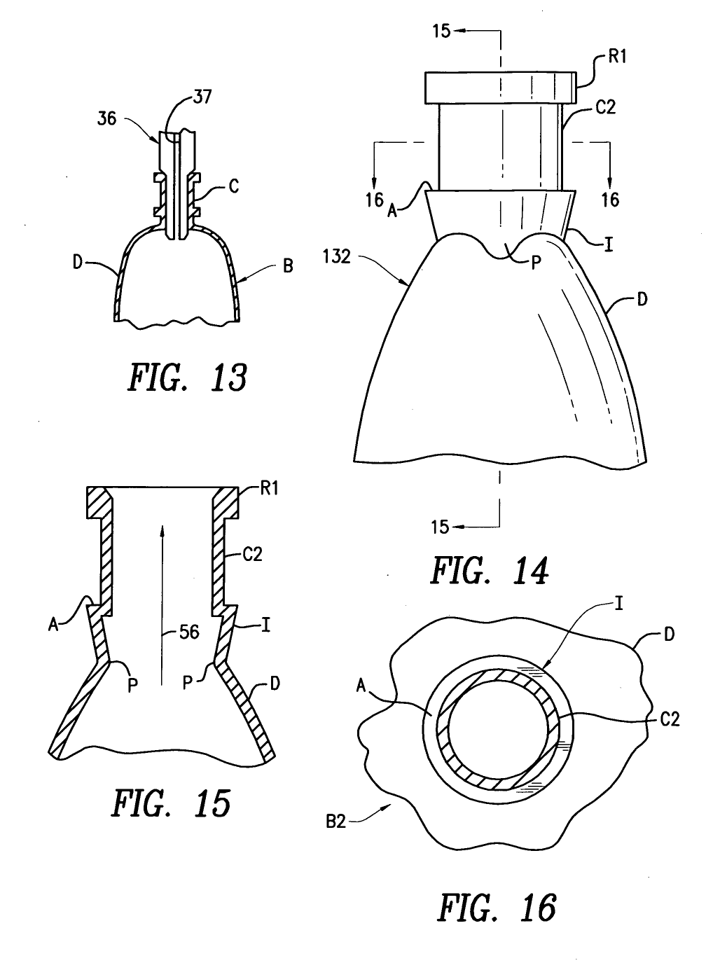

Referring again to FIG. 9, it has been found that upon the completion of the forming of the breathing bag B, and upon the opening of the main molds 18 and 20 for the withdrawal of the combination blowing and forming member 36 from the connector portion C as described above, as illustrated in FIG. 13, sufficient adhesion can exist between the breathing bag connector potion C and the combination blowing and forming member 36 as to require unwanted additional manual manufacturing step(s), with their unwanted attendant additional manufacturing cost, for the removal of the connector portion C, and thereby the breathing bag B, from the member 36. A further or alternate embodiment of a breathing bag embodying the present invention and, which overcomes this problem, is shown in FIGS. 14-16 and indicated by general alphabetical designation B2.

It will be generally understood, and referring to FIGS. 14-16, that breathing bag B2 is substantially the same as breathing bag B of FIGS. 6 and 6A, except that breathing bag B2 does not include the annular ridge R2 of breathing bag B(FIG. 6A), and instead includes an intermediate inverted truncated conical portion indicated by general alphabetical designation I in FIGS. 14-16. As shown in FIGS. 14-16, the inverted truncated conical portion I includes, or provides, an annular surface A disposed perpendicular, or at least substantially perpendicular, to the connector portion C2. It will be further generally understood that the breathing bag B2 of FIGS. 14-16 includes integrally formed connector portion C2, distensible bag portion D and inverted truncated conical portion I intermediate the connector portion C2 and the distensible bag portion D; as taught above with regard to the connector portion C of the breathing bag B, the connector portion C2 of the breathing bag B2 is sufficiently thick or rigid to connect directly to a male fitting.

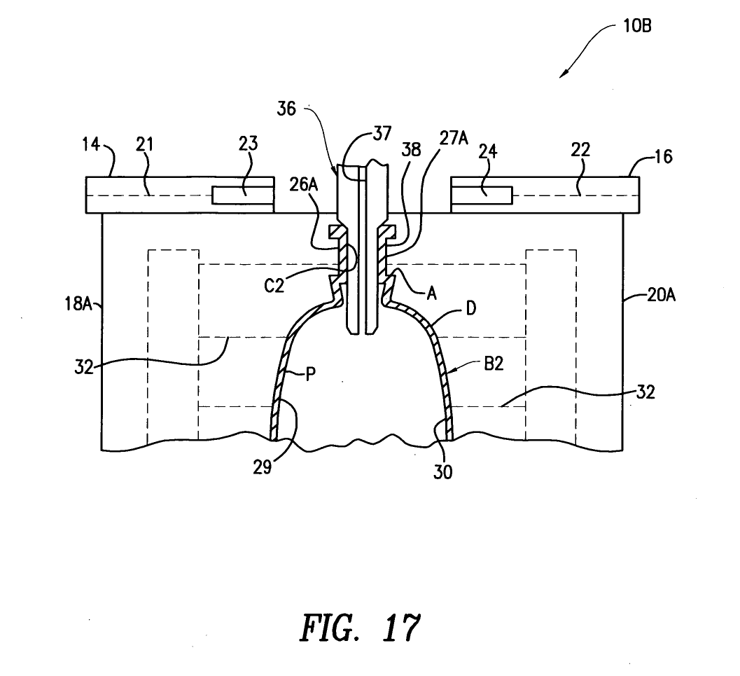

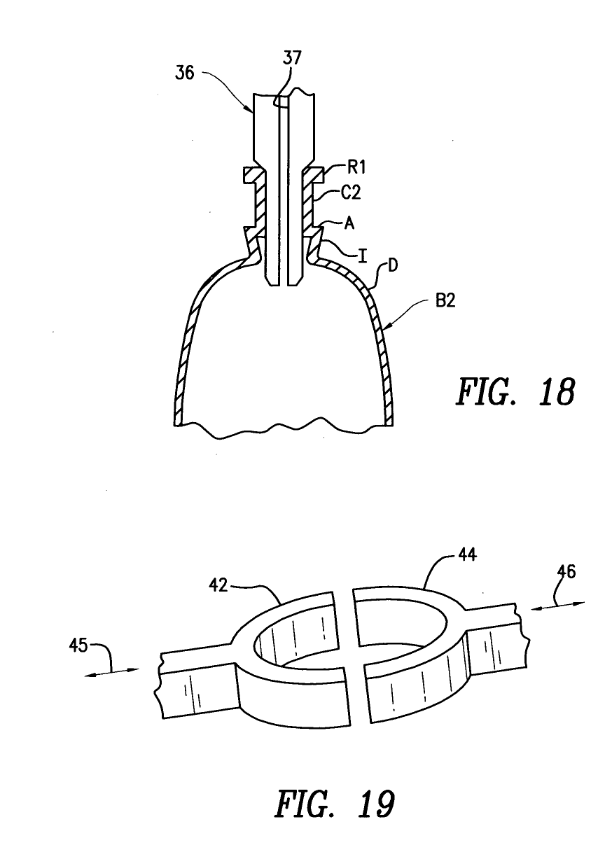

It will be still further understood that the process for manufacturing the breathing bag B2 may be substantially the same as the process for manufacturing the breathing bag B described above in connection with apparatus 10 shown in FIGS. 7-9. Accordingly, apparatus suitable for manufacturing the breathing bag B2 is shown in FIG. 17 and indicated generally by numerical designation 10B; apparatus 10B is substantially the same as apparatus 10 shown in the upper portion of FIG. 9 except that the connector forming portions 26A and 27A of the main molds 18A and 20A, FIG. 17, are complementary in shape to the connector portion C2 and the inverted truncated conical portion I of the breathing bag B2 shown in FIGS. 14-16. Upon the completion of the manufacture of the breathing bag B2, the main molds 18A and 20A are opened and the connector portion C2 of the breathing bag B2 is adhered to the combination blowing and forming member 36 as shown in FIG. 18. In the manufacturing process for manufacturing the breathing bag B2, it will be understood that the compressive forces applied to form the connector portion C2 of the bag B2 are sufficient to assure that the connector portion C2 adheres or sticks to the combination blowing and forming member 36 upon the main molds 18A and 18B being opened.

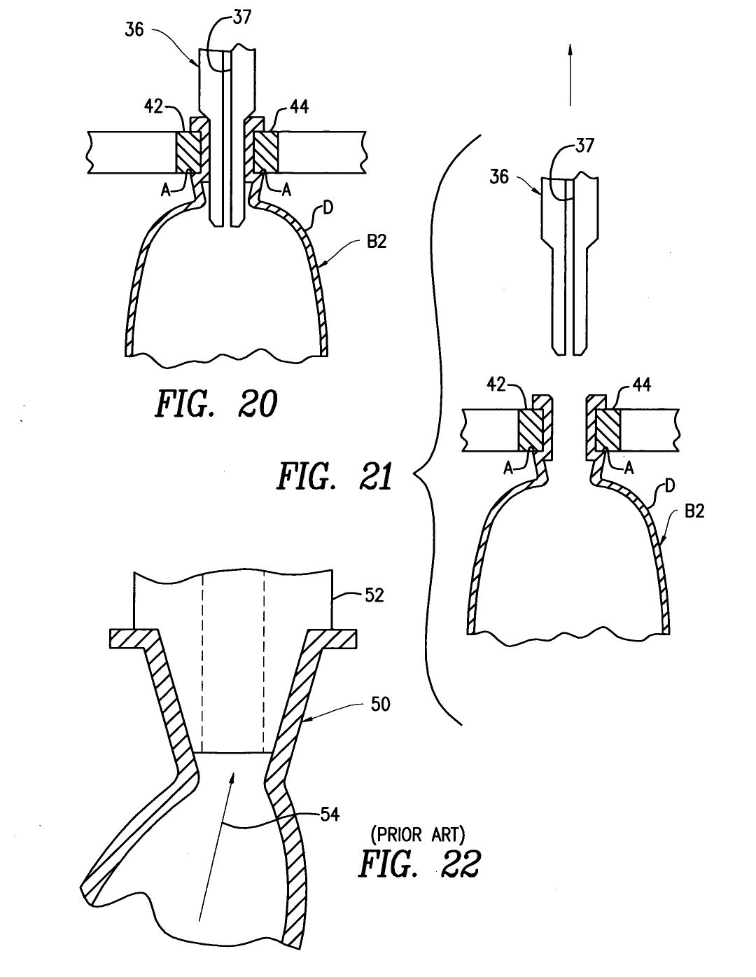

A pair of suitable, opposed semi-circular engaging and transfer members 42 and 44, shown in FIG. 19, may be provided, and which members 42 and 44 are mounted for reciprocating movement towards and away from each other as indicated by the double headed arrows 45 and 46 in FIG. 19. As shown in FIG. 20, the members 42 and 44 are advanced into engagement with the connector portion C2 of the breathing bag B2 with the undersides of the members 42 and 44 engaging the annular surface A of the intermediate inverted truncated conical portion I of the bag B2. The members 42 and 44 maintain the connector portion C2, and thereby the breathing bag B2 stationary, particularly by engaging the annular surface A of the intermediate truncated conical portion I, as the combination blowing and forming member 36 is moved upwardly, as indicated by the arrow 48 in FIG. 21, which permits the adhesion between the connector portion C2 and the member 36 to be broken and the member 36 to be withdrawn from the connector portion C2. The annular surface A of the inverted truncated conical portion I acts, in essence, as a hold down surface which permits the members 42 and 44 to hold the connector portion C2, and thereby the breathing bag B2, down as the combination blowing and forming member 36 is withdrawn from the connector portion C2. Thereafter, the members 42 and 44 may transfer the separated breathing bag B2 to, for example, a conveying station whereupon the members 42 and 44 are moved away from each other and the separated breathing bag B2 is deposited at the conveying station and conveyed to, for example, a packing station.

The intermediate inverted truncated conical portion I, FIG. 14, also serves the same function as the ridge R2 provided on the breathing bag B of FIG. 6A, namely it provides a feature for gripping by the hand of a person to facilitate the removal of the connector portion C2 from a male fitting.

As illustrated in FIG. 22, while an anesthesiologist is administering anesthesia to a patient using a prior art breathing bag 50 connected to a male fitting 52, and while the eyes of the anesthesiologist may be fixed on the gauge or display of an anesthesia machine, the anesthesiologist may inadvertently bend the breathing bag 50 at an angle, particularly an acute angle, with respect to the male fitting 52 which can occlude, or at least partially occlude, the internal airway, indicated by the arrow 54 in FIG. 22, which occlusion is most undesirable. The breathing bag B2, and referring again to FIGS. 14 and 15, may be provided with a pair of diametrically opposed and inwardly extending nubs or projections P-P which prevent the occlusion of the internal airway provided in the breathing bag B2, and which airway is indicated by the arrow 56 in FIG. 15, upon the breathing bag B2 being bent with respect to a male fitting to which the breathing bag is attached.

It will be understood that many variations and modifications may be made in the present invention without departing from the spirit and scope thereof.

What is claimed is:

1. A breathing bag for being connected directly to a male fitting, comprising:
integrally formed connector portion, distensible bag portion and an inverted truncated conical portion intermediate said connector portion and said distensible bag portion, said connector portion sufficiently rigid to connect directly to the male fitting.

2. The breathing bag according to claim 1 wherein said inverted truncated conical portion provides an annular surface substantially perpendicular to said connector potion.

3. The breathing bag according to claim 1, wherein said connector portion is generally cylindrical and includes an outer portion provided with an integrally formed, radially outwardly extending and generally annular ridge.

4. The breathing bag according to claim 2 wherein said connector portion is generally cylindrical and includes an outer portion provided with an integrally formed, outwardly extending and generally annular ridge and wherein said annular ridge and said annular surface are parallel and spaced apart.

5. The breathing bag according to claim 3, wherein said generally annular ridge is generally rectangular in transverse cross-section.

6. The breathing bag according to claim 1 wherein said connector portion said distensible bag portion and said inverted truncated conical portion provide said breathing bag with an internal airway, wherein a transition portion is provided intermediate said distensible bag portion and said inverted truncated conical portion, and wherein said transition portion is provided with a pair of diametrically opposed, inwardly extending projections for preventing occlusion of said airway.
